(19) United States
(12) Patent Application Publication
(10) Pub. No.: US 2023/0233720 A1
(43) Pub. Date: Jul. 27, 2023

(54) SYSTEM FOR PRETREATING AND DISINFECTING WASTE

(71) Applicant: BERTIN TECHNOLOGIES, Montigny Le Bretonneux (FR)

(72) Inventors: Aurélien BATAILLE, Lescure d'albigeois (FR); Thibault SENTENAC, Voisins-le-Bretonneux (FR); Jean ROCH, Simiane-collongue (FR)

(21) Appl. No.: 18/011,009

(22) PCT Filed: Jun. 8, 2021
(86) PCT No.: PCT/EP2021/065272

§ 371 Date: Dec. 16, 2022

(30) Foreign Application Priority Data
Jun. 17, 2020 (FR) FR2006320

(51) Int. Cl. A61L 2/12; A61L 11/00; A61L 2/20

(52) U.S. Cl. CPC A61L 2/12; A61L 11/00; A61L 2/202; A61L 2202/121

(57) ABSTRACT

A system for pretreating and disinfecting waste, wherein the system includes a transport line configured to move the waste, and including a conveying device, a heating device configured to heat a part at least of the waste when the waste moves along a part at least of the transport line, and in that the system includes a tunnel hermetically sealing a part at least of the waste transport line, and in that the system further includes an ozone injection device configured to apply ozone on a part at least of the waste when the waste moves along a part at least of the transport line.

### TECHNICAL FIELD OF THE INVENTION

The present invention relates to the field of waste, and particularly Potentially Infectious Medical Waste (PIMW). It is particularly advantageously applied in the field of PIMW pretreatment and disinfection.

### PRIOR ART

There are very many PIMW pretreatment and disinfection systems in the prior art. Generally, these systems comprise a waste shredding device and a waste heating device, and, frequently, the two devices form a single device for heating during shredding for example. Thus, the document WO2013110900 A1 is known, which describes a PIMW pretreatment and disinfection machine comprising a tank mounted on a frame, shredding means mounted at the bottom of the tank and a device for heating waste with a view to the disinfection thereof. This heating device comprises a microwave generator connected by a waveguide to the tank, the waste thus being heated in the tank where it is shredded.

It is in fact common, for the purposes of compactness and efficiency, that the heating be generated by microwaves and that the waste be heated in the shredding tank.

Nevertheless, one of the main problems that pretreatment and disinfection systems can encounter relates to the disinfection of the areas of the system in contact with the waste prior to the pretreatment and disinfection thereof.

In the prior art, it is necessary for example to dismantle the devices for feeding or discharging waste to perform the disinfection thereof, on a recurrent basis.

An aim of the present invention is therefore that of proposing a solution for this problem.

Moreover, a medical waste treatment system is known from patent publication WO 2013/096782 A2. The technique described comprises a waste transport line. However, the management of this line, particularly of the atmosphere of the chamber thereof, is not optimal.

The other subject matter, features and advantages of the present invention will become apparent on studying the following description and the accompanying drawings. It is understood that other advantages can be incorporated.

### SUMMARY OF THE INVENTION

To achieve this aim, according to an embodiment, a system for pretreating and disinfecting waste is provided, particularly potentially infectious medical waste, characterized in that it comprises:
a. a transport line configured to move the waste between an intake area and a discharge area of the system, and comprising a conveying device, the conveying device being configured to move the waste along a part at least of the transport line,
b. a heating device configured to heat a part at least of the waste when the waste moves along a part at least of the transport line,
   and in that the system comprises a tunnel hermetically sealing a part at least of the waste transport line, the conveying device being housed in said tunnel, said tunnel comprising a waste entry and a waste exit, and
   in that the system further comprises an ozone injection device configured to apply ozone on a part at least of the waste when the waste moves along a part at least of the transport line.

The present solution makes it possible to optimize the disinfection waste, in particular PIMW. It makes it possible to ensure that the waste at the exit is inert and considered as household waste.

The present invention makes it possible to disinfect at least a part of the elements making up the system, in particular the tunnel. It enables a user to work safely during repairs for example.

Indeed, ozone enables disinfection of various areas where it circulates in the system. It makes it possible to disinfect for example the shredder, or the feed hopper, or the tunnel efficiently and during the use of the system.

Ingeniously, ozone makes it possible to disinfect the entire tunnel. Thus, at the end of production, the present invention makes it possible to disinfect the tunnel for example by continuing the injection of ozone to disinfect the entire tunnel and enable a user to perform, if required, maintenance operations in complete safety.

The present solution makes it possible to reduce or eliminate odors through the oxidizing capacity of ozone, which enables easier equipment integration. Finally, it makes it possible to remove, at least partially, aero-contaminants which represent a substantial contamination risk when shredding containers/bags of infectious hospital waste for example.

Ingeniously, the present solution synergistically combines heat and ozone in order to obtain additional waste disinfection. Ozone has a surface disinfecting capacity and penetrates inside the waste material according to the texture thereof, and the heat enables a temperature rise of the waste and the disinfection thereof.

Another aspect relates to a method for pretreating and disinfecting waste, particularly potentially infectious medical waste, comprising at least the following steps implemented by the system according to the present solution:
a. Moving the waste in a tunnel by a conveying device along a transport line, the tunnel hermetically sealing a part at least of the waste transport line;
b. Heating the waste moving in the tunnel by at least one heating device;
c. Before or during the two preceding steps, applying ozone on a part at least of the waste moving along the transport line by an ozone injection device.

### BRIEF DESCRIPTION OF THE FIGURES

The aims, subject matter, and the features and advantages of the invention will become more apparent from the detailed description of an embodiment thereof which is illustrated by the following accompanying drawings wherein:

FIG. 1 schematically represents a pretreatment and disinfection system according to an embodiment of the present invention.

The drawing is given by way of example and is not restrictive in respect of the invention. It represents a schematic conceptual representation intended to facilitate the understanding of the invention and is not necessarily to scale in respect of practical applications. In particular, the dimensions are not representative of reality.

### DETAILED DESCRIPTION

Before commencing a detailed review of embodiments of the invention, optional features which may be used in combination or alternatively are listed hereinafter:

According to an example, the system comprises at least one extraction device configured to extract, from a first area of the tunnel, a portion at least of the atmosphere contained in the tunnel.

This enables management of the quantity of ozone present in the tunnel, by analyzing parameters such as hydrometry/temperature, etc.

According to an example, the extraction device is configured to analyze at least one parameter of the atmosphere taken from at least: hydrometry, temperature, ozone concentration.

According to an example, the extraction device is configured to reinject at least partially said atmosphere portion from a second area of the tunnel.
This makes it possible to preheat the waste and reuse a part at least of the injected ozone.
This makes it possible to reduce the heating power required to reach a predetermined disinfection temperature.

According to an example, the extracted atmosphere portion is a gas mixture comprising a part at least of the ozone injected by the injection device and at least one other gas.

According to an example, the first area is located at the tunnel exit and the second area is located at the tunnel entry.

This makes it possible to preheat the waste before it is heated by the heating device.

According to an example, the system comprises at least one device for stirring the waste disposed upstream from the tunnel entry along a direction of waste transport passing through the entry then through the exit.
This makes it possible to stir, or shred the waste before it enters the tunnel, in order to increase the exchange surface area with the ozone and the heat generated by the heating device.

According to an example, the stirring device is a waste shredding device.

According to an example, ozone is injected into the stirring device and/or upstream from the stirring device along a direction of waste transport passing through the entry then through the exit.
This makes it possible to disinfect the stirring device and remove any aero-contaminant generated in the stirring, preferably grinding, phase.

According to an example, the system comprises at least one feed hopper disposed upstream from the stirring device along a direction of waste transport passing through the entry then through the exit, and the injection device is configured to inject ozone at the feed hopper.
This makes it possible to partially disinfect at least the waste in the feed hopper before stirring, preferably before shredding.

According to an example, the injection device is configured to inject ozone at the waste intake area.
This makes it possible to disinfect the elements of the transport line from the intake area to the tunnel exit.

According to an example, the injection device is configured to inject ozone at the tunnel entry.

According to an example, the transport line has an inclined portion relative to a horizontal plane such that a part of this inclined portion is located at a lower height than the height at which another part of this inclined portion with respect to said horizontal plane is located.

According to an example, the conveying device is inclined with respect to a horizontal plane in the direction of movement of the waste.

According to an example, the conveying device comprises at least a first conveyor and a second conveyor.

According to an example, the first conveyor and the second conveyor have a height offset H relative to each other such that the waste arriving at the end of travel of the first conveyor drops onto the second conveyor.
This enables a stirring effect of the waste in the tunnel in order to change the orientation thereof with respect to the ozone and also to the heating device.

According to an example, the system comprises at least one sensor configured to measure the ozone concentration in the atmosphere outside the tunnel.
This makes it possible to ensure safety and assess an ozone leak risk.

According to an example, the system comprises at least one sensor configured to measure the ozone concentration in the tunnel and preferably the hydrometry level.
This makes it possible to regulate the quantity of ozone present in the tunnel.

According to an example, the device for injecting ozone is configured so that the ozone concentration in the tunnel is at least equal to a few grams per m3.

According to an example, the heating system comprises at least one microwave generator.

According to an example, the heating system comprises a plurality of microwave generators.

According to an example, the heating device comprises at least a plurality of microwave generators with associated waveguides to transmit the microwaves generated in the microwave generator into the tunnel.

According to an example, the heating device comprises at least a plurality of microwave generators and a plurality of waveguides configured to transmit the microwaves generated by the microwave generator into the tunnel, preferably each waveguide of the plurality of waveguides being associated with a microwave generator of the plurality of microwave generators.

According to an example, the heating device comprises at least a plurality of microwave generators and a plurality of associated waveguides configured so that the microwaves generated are distributed along the transport line in the tunnel.

According to an example, the heating device is configured so that the microwaves generated are distributed along the transport line in the tunnel. Advantageously, the microwaves have a core disinfecting capacity to spread from the center of the waste material outward. This makes it possible to heat the waste along the tunnel homogeneously.

According to an example, the step of applying ozone is performed before and/or at the same time as the heating step. Advantageously, the ozone has a surface disinfecting capacity. Thus by associating heating, preferably by microwaves for core disinfection of the waste, and the application of ozone, for surface disinfection, the disinfection performances are optimized.

According to an example, the method comprises, prior to the circulation step, a step of stirring, preferably shredding the waste, by a stirring device, preferably by a shredding device.

According to an example, the method comprises at least one step of extracting, from a first area of the tunnel, a portion at least of the atmosphere contained in the tunnel.

According to an example, the method comprises, after the extraction step, at least one step of reinjecting from a second area of the tunnel said portion removed from the atmosphere.

According to an example, the second area of the tunnel comprises at least one air-lock, preferably at least one intermediate hopper, and the extraction is configured to reinject at least partially said atmosphere portion in said air-lock.

The present invention is applicable in the field of the pretreatment and disinfection of waste, and in particular Potentially Infectious Medical Waste (PIMW). Ingeniously, the present invention enables the treatment of a large quantity of water, and preferably in succession, and advantageously continuously. The present invention further enables disinfection of the waste and also of a part at least of the pretreatment and disinfection system, simply, quickly and reliably.

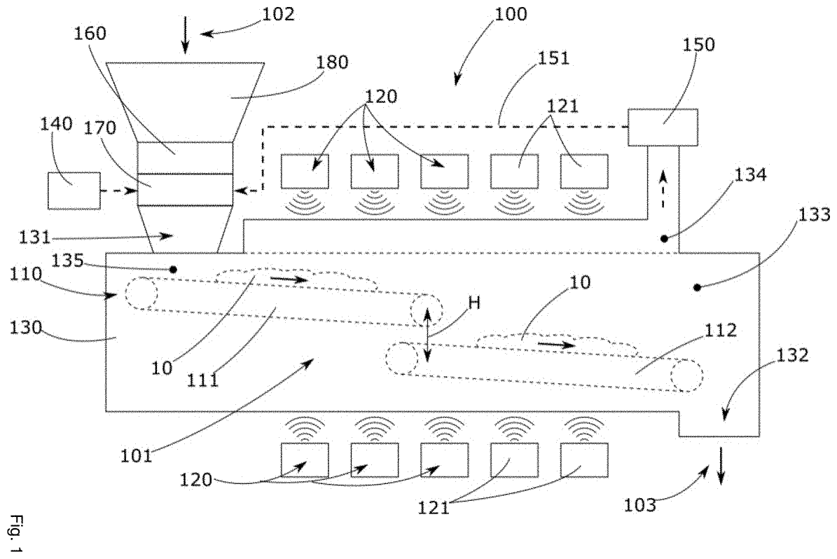

Indeed, according to the present invention, and as described in FIG. 1, the waste 10 pretreatment and disinfection system 100 comprises:
a. A waste 10 transport line 101;
b. Preferably, a heating device 120;
c. A tunnel 130 hermetically sealing a part at least of the transport line 101;
d. An ozone injection device 140;
e. Preferably, an extraction device 150;
f. Preferably, a stirring device 160.

According to an embodiment, the waste 10 transport line 101 is configured to move the waste 10 through the pretreatment and disinfection system 100. The waste 10 transport line 101 is configured to move the waste 10 between an intake area 102 and a discharge area 103 of the system 100 according to the present invention. A part of the transport can be sequential, for example moving the waste in successive batches. This can be the case during shredding for example. Another part of the line 101 can perform continuous movement. This can be the case during a conveying operation.

This transport line 101 can preferably comprise a waste 10 conveying line 110. Advantageously, the conveying device 110 is configured to move the waste 10 along a part at least of the transport line 101. According to an embodiment, the conveying device can comprise one or more conveyors. Typically, such a conveyor may comprise a belt on the top surface whereof the waste is disposed in transit, and means for driving the belt, for example in the form of rollers; the belt forms a continuous assembly, rotating in a loop.

In FIG. 1, and by way of non-limiting example, the conveying device 110 comprises a first conveyor 111 and a second conveyor 112. These conveyors 111 and 112 can be of the same type or have a different conveying mechanism. Ingeniously, the first conveyor 111 and the second conveyor 112 are disposed relative to one another so as to enable stirring of the waste, in particular a spatial repositioning of the waste 10. This thus makes it possible to move the waste 10 and improve the contacting thereof with the atmosphere 133 of the tunnel 130 and therefore with the ozone. This also makes it possible to improve the homogeneity of the heating of the waste 10 when moving in the tunnel 130.

For example, and as illustrated in FIG. 1, the first conveyor 111 and the second conveyor 112 can be disposed relative to one another such that when the waste 10 arrives at the end of travel of the first conveyor 111, it falls onto the second conveyor 112, this drop enabling the stirring of the waste 10. Thus, the end of the first conveyor 111 can be disposed plumb with the start of the second conveyor 112, for example, preferably according to a height dimension H greater than 20 cm, advantageously than 50 cm.

Thus, according to a preferred embodiment, at the halfway point of the waste 10 in the tunnel 130, the first conveyor 111 offloads the waste onto the second conveyor 112, in order to perform a mixing function and optimize the penetration of the ozone and microwaves into the waste. This thus makes it possible to increase the efficiency of the pretreatment and disinfection.

Advantageously, and as illustrated in FIG. 1, the first conveyor 111 and/or the second conveyor 112 can be inclined with respect to each other and/or with respect to the horizon.

Preferably, this inclination is configured so that the waste exit is lower in altitude than the entry thereof on the conveying device. The inclination of the conveyors can be identical.

Advantageously, to prevent a gravitational descent of the waste on the conveyors, the inclination relative to the horizontal can be chosen less than 10°.

According to an embodiment, the heating device 120 is configured to heat a part at least of the waste 10 when the waste 10 moves along a part at least of the transport line 101, preferably in the tunnel 130. Advantageously, this heating device 120 can comprise one or more microwave generators, and/or comprise one or more waveguides. Preferably, the heating device 120 is configured to heat a part at least of the waste 10 to a temperature greater than 100° C.

Preferably, at least a part of the heating is performed during the conveying. This does not exclude, additionally or alternatively, that the heating be produced at other locations of the transport line 101, for example during shredding.

In FIG. 1, by way of non-restrictive example, the heating device 120 comprises a plurality of microwave generators 121 disposed along the tunnel 130 so as to enable a homogeneous irradiation of a part at least of the waste 10 passing through the tunnel 130.

Advantageously, and as illustrated in FIG. 1, the plurality of microwave generators 121 can be distributed at different locations around the conveying device 110 so as to irradiate from above and below, and/or from one side and the other, the waste 10 during the transport thereof in a part at least of the tunnel 130.

According to an embodiment, the waste 10 tunnel 130 comprises a waste 10 entry 131 and a waste 10 exit 132. Preferably, the conveying device 110 is housed in the tunnel 130. Ingeniously, the first conveyor 111 is positioned so as to directly receive the waste 10 entering, preferably dropping, via the entry 131 of the tunnel 130.

Ingeniously, the second conveyor 112 is positioned so as to discharge, preferably drop, the waste 10 toward the exit 132.

Advantageously, this tunnel 130 defines a hermetic volume around a part of the transport line 101.

Thus, and preferably, the atmosphere 133 present in this volume is intended to be confined therein relative to the exterior of the tunnel 130.

According to an embodiment, the ozone injection device 140 is configured to inject ozone into the atmosphere 133 of the tunnel 130, preferably to inject a predetermined quantity of ozone into the atmosphere 133 of the tunnel 130. Advantageously, the injection device 140 is configured to apply ozone, preferably a predetermined quantity of ozone, on a part at least of the waste 10 when it is in the tunnel 130.

Preferably, the injection device 140 is configured to inject ozone at the entry 131 of the waste 10 into the tunnel 130. In the present description, the expression “at” can be understood to mean “inside”; for example, the injection of a gas at a device means the injection of said gas into a part at least of a volume defined by a part at least of said device.

According to an embodiment, the injection device 140 is configured to inject ozone into the first third of the length of the transport line 101 located in the tunnel 130, preferably in the first quarter thereof.

As illustrated in FIG. 1, the injection device 140 can also inject ozone at an air-lock 170 separating the intake area 102 from the entry 131 of the tunnel 130.

According to an embodiment, the stirring device 160 is disposed upstream from the entry 131 of the tunnel 130, for example before the air-lock 170. In the present description, the direction of waste transport is considered to be through the entry 101 then through the exit 102 of the system.

This stirring device 160 can for example be a shredder. This stirring device 160 is configured to stir, or to shred, the waste 10. Preferably, the stirring device 160 is used to shred the waste 10 before it enters the tunnel 130.

Advantageously, and according to an embodiment, ozone is injected at the air-lock 170, when the latter is closed, preferably by means of a lid for example, in order to disinfect the aero-contaminants generated during stirring, preferably shredding of the waste.

According to a further embodiment, compatible with the preceding one, ozone is injected downstream from the stirring device 160 as illustrated in FIG. 1. It will be noted that the air-lock 170 can separate the stirring device 160 from the tunnel 130, and preferably from the ozone injection point by the injection device 140.

According to an embodiment, the air-lock 170 can comprise a hatch opening onto the entry 131 of the tunnel 130, or forming partially at least said entry 131 of the tunnel 130. This hatch is advantageously hermetic when it is closed.

According to an embodiment, the air-lock 170 comprises an intermediate hopper. Preferably, ozone can be injected by the injection device 140 at this intermediate hopper, which is used to inject, preferably continuously, ozone into the tunnel 130, and therefore disinfect, preferably continuously, the waste moving in the tunnel 130. Preferably, according to an embodiment, the injected ozone mixed with the atmosphere 133 of the tunnel 130 circulates in the tunnel 130 by means of the extraction device 150. Ingeniously, once the extraction is stopped, the injection of ozone stops automatically.

According to a further embodiment, the ozone is injected into a volume defined by a part at least of the stirring device 160. Preferably, the ozone is injected before or during the stirring of the waste 10. According to an embodiment, the stirring device comprises a tank, advantageously hermetic, intended to contain the waste 10 during the stirring thereof. Said volume wherein the ozone is injected can be defined by this tank. This makes it possible to expose the waste 10 to the ozone directly in the tank of the stirring device 160, and thus disinfect the inside of the tank.

According to a further embodiment, a feed hopper 180 is disposed downstream from the stirring device 160. This feed hopper 180 comprises a hermetic hatch for rendering the interior of the stirring device 160 hermetic relative to the exterior. According to this embodiment, the injection device 140 can then be configured to inject ozone into the feed hopper 180 such that the waste is in contact with the ozone when being stirred, preferably shredded, by the stirring device 160.

This makes it possible to disinfect the waste 10 in the feed hopper 180 before shredding, preferably at ambient temperature. This also makes it possible to disinfect the inside of the stirring device 160 and the feed hopper 180, preferably continuously, in particular before opening the air-lock 170 of the tunnel 130. Finally, this makes it possible to remove the aero-contaminants in the stirring volume. Preferably, ozone is injected only when the feed hopper 180 is closed, i.e., when the lid thereof to the outside is closed.

Advantageously, ozone is injected upstream from the stirring device 160.

According to an embodiment, the feed hopper 180 comprises a lid, preferably motorized. In the case where ozone is injected partially at least at the feed hopper 180, i.e., in a volume defined partially at least by the feed hopper 180, the injection device 140 is configured to only inject ozone at the feed hopper 180 when the lid is closed.

According to a preferred embodiment, ozone is injected both upstream and downstream from the stirring device 160, preferably at the stirring device 160 and downstream from the stirring device 160. According to this embodiment, the ozone injection device 140 is configured to inject ozone at the feed hopper 180, or in the tank of the stirring device 160, before or while the waste is stirred, preferably shredded, and to inject ozone at the intermediate hopper, i.e., at the air-lock 170, in order to supply the atmosphere of the tunnel 130 with ozone.

Ingeniously, the ozone injected at the intermediate hopper comes, partially at least, from the reinjection circuit 151.

Advantageously, the system 100 is configured to receive ozone upstream and downstream from the stirring device 160.

According to an embodiment, the extraction device 150 is configured to extract a portion at least of the atmosphere 133 of the tunnel 130. In particular, the extraction device 150 is configured to extract a gaseous mixture of ozone and water vapor. This water vapor comes preferably from heating the waste 10. Indeed, on heating, a part at least of the waste 10 produces water vapor.

On heating, the waste 10 generates vapor, which will then be extracted, partially at least, and reinjected at the entry 131 of the tunnel 130 to preheat this waste 10. This preheating facilitates the temperature rise of the waste 10 moving in the tunnel 130. This makes it possible to reduce the heating power required to obtain the disinfection of the waste 10. This water vapor and a part of the ozone then form a hot gaseous mixture. Advantageously, this hot gaseous mixture, preferably during the waste treatment, has a temperature greater than 70° C., preferably than 85° C. and advantageously than 100° C.

Ingeniously, the extraction device 150 is also configured to enable the reinjection of said removed portion of atmosphere 133 in the tunnel 130. Advantageously, the extraction device 150 is configured to extract said removed portion at a first area 133 of the tunnel 130, preferably close to or merged with the exit 132 of the tunnel 130. And preferably, the extraction device 150 is configured to enable the reinjection of said removed portion at a second area 134 of the tunnel 130, preferably close to or merged with the entry 131 of the tunnel 134, or with the ozone injection area.

According to an embodiment, said removed portion is reinjected by the injection device 140.

Note will be taken in FIG. 1 of the reinjection circuit 151 which enables the hot gaseous mixture to flow from the first area 133 of the tunnel 130 to the second area 134 of the tunnel 130.

According to an embodiment, this reinjection circuit fluidically connects the extraction device 150 to the injection device 140.

Advantageously, extracting this hot gaseous mixture at the end of the tunnel 130 and reinjecting it at the start of the tunnel 130 makes it possible to preheat the waste 10 entering the tunnel 130 and thus makes it possible to reduce the heating power required supplied by the heating device 120.

Ingeniously, the hot gaseous mixture is reinjected either at the feed hopper, or at the stirring device, or at the air-lock 170, so as to preheat the waste 10. Therefore, this makes it possible to limit the heating power required from the heating device 120 to obtain the disinfection stage, preferably equal to 100° C. According to this embodiment, the previously injected ozone is thus mixed with this hot gaseous mixture comprising water vapor.

Thus, the present invention makes it possible when the waste 10 is moving in the tunnel 130, to expose this waste 10 to a predetermined temperature and to a predetermined ozone level, so as to enable the pretreatment and disinfection thereof and the disinfection inside the tunnel 130.

The present invention also relates to a method for the pretreatment and disinfection of waste, in particular potentially infectious medical waste. This method comprises at least the following steps:
a. Preferably stirring waste 10 by a stirring device 160 disposed upstream from the entry 131 of the tunnel 130;
b. Moving the waste 10 in the tunnel 130 by the conveying device 110 along the transport line 101;
c. Preferably, heating a part at least of the waste 10 moving in the tunnel 130 by the heating device 120;
d. Before or during the preceding steps, applying ozone on a part at least of the waste 10 by the injection device 140.

According to an embodiment, ozone is applied before or while the waste 10 is heated.

According to an embodiment, the method also comprises a step of extracting by the extraction device 150, from a first area 133 of tunnel 130, a portion at least of the atmosphere 133.

Advantageously, the method can comprise, after the extraction step, at least one step of reinjecting from a second area 134 of the tunnel 130 said portion removed from the atmosphere 133.

According to a preferred embodiment, the injection of ozone takes place in the stirring volume of the waste 10 in the stirring device 160. Preferably, the injection of ozone takes place before the step of stirring the waste 10 in the stirring device 160.

Advantageously and preferably, the reinjection of the extracted portion of atmosphere 133 takes place at an intermediate hopper disposed between the stirring device 160 and the tunnel 130.

Thus, the present invention ingeniously uses two disinfection methods, one based on heating the waste and the other on using ozone. These two methods are preferably, but not restrictively, configured to cooperate together. Ozone has a surface disinfectant capacity and penetrates inside the waste according to the textures thereof. Microwaves have a core disinfecting capacity to spread from the center of each waste material outward.

Particularly advantageously, ozone makes it possible to disinfect partially at least the inside of the tunnel. Thus, for example, when there is no more waste to treat, it is possible to disinfect the inside of the tunnel by creating an injection/extraction/ozone reinjection loop in a closed circuit.

This then enables the user to perform if required maintenance operations in complete safety.

Finally, ozone has the advantage of oxidizing a part at least of the odorous molecules generated by the waste, which reduces the nuisance of these odors.

Thus, the present invention makes it possible to:
a. Disinfect waste with a physicochemical method, ozone and heating, which makes it possible to convert potentially infectious waste into disinfected inert waste;
b. Disinfect the equipment at end of cycle, which increases safety for users during maintenance;
c. Reduce odor thus enabling easier equipment integration.

It should be noted that the present invention can also comprise a safety device, in particular in compliance with regulations in respect of ozone emissions in air, in order to maintain a predetermined average exposure value, for example over an 8-hour period, the predetermined average exposure value of ozone having to be less than or equal to around 0.1 ppm.

Thus, the pretreatment and disinfection system can comprise a safety device configured to measure, advantageously in real time, the ozone concentration in and/or around the tunnel.

Preferably, the safety device is configured to manage the injection device, or also the extraction device.

The pretreatment and disinfection system, in particular the tunnel, is preferably configured to be leak-tight so as to reduce, or prevent, any risk of ozone leakage into the atmosphere outside the tunnel. Similarly, the tunnel is preferably configured to reduce, or prevent any microwave leakage.

Ingeniously, the pretreatment and disinfection system according to the present invention thus comprises warning sensors disposed around the tunnel, and more generally all of the devices of the system.

Preferably, the pretreatment and disinfection system comprises at least one air suction device disposed around the tunnel, and advantageously all of the equipment of the system capable of having an ozone leak. This makes it possible to draw in any ozone leaks.

Finally, the present invention makes it possible to treat a large quantity of waste reliably, quickly and efficiently.

This invention is not restricted to the embodiments described above and extends to all embodiments covered by the claims.

### REFERENCES

10 Waste
100 Pretreatment and disinfection system
101 Transport line
102 Intake area
103 Discharge area
110 Conveying device
111 First conveyor
112 Second conveyor
120 Heating device
121 Microwave generator
130 Tunnel
131 Tunnel entry
132 Tunnel exit
133 Tunnel atmosphere
134 First area of tunnel
135 Second area of tunnel
140 Injection device
150 Extraction device
151 Reinjection circuit
160 Stirring/Shredding device
170 Air-lock or intermediate hopper
180 Feed hopper
H Height offset

1. A system for pretreating and disinfecting waste, said system comprising:
a transport line configured to move the waste between an intake area and a discharge area of the system, and comprising a conveying device, the conveying device being configured to move the waste along a part at least of the transport line,
a heating device configured to heat a part at least of the waste when the waste moves along a part at least of the transport line,
and in that the system comprises a tunnel hermetically sealing a part at least of the waste transport line, the conveying device being housed in said tunnel, said tunnel comprising a waste entry and a waste exit, and
in that the system further comprises an ozone injection device configured to apply ozone on a part at least of the waste when the waste moves along a part at least of the transport line,
the system comprising at least one extraction device configured to extract, from a first area of the tunnel, a portion at least of the atmosphere contained in the tunnel,
the extraction device being configured to reinject at least partially said portion of atmosphere from a second area of the tunnel.

2. The system according to claim 1 wherein the first area is located at the tunnel exit and the second area is located at the tunnel entry.

3. The system according to claim 2 wherein the second area of the tunnel comprises at least one air-lock, and wherein the extraction device is configured to reinject at least partially said portion of atmosphere into said air-lock.

4. The system according to claim 3 wherein the air-lock comprises at least one intermediate hopper.

5. The system according to claim 1 wherein the extracted portion of atmosphere is a gas mixture comprising a part at least of the ozone injected by the injection device and at least one other gas.

6. The system according to claim 1 comprising at least one device for stirring the waste disposed upstream from the tunnel entry along a direction of waste transport passing through the entry then through the exit.

7. The system according to claim 6 wherein the stirring device is a waste shredding device.

8. The system according to claim 6 wherein the ozone is injected into the stirring device and/or upstream from the stirring device along a direction of waste transport passing through the entry then through the exit.

9. The system according to claim 6 comprising at least one feed hopper disposed upstream from the stirring device along a direction of waste transport passing through the entry then through the exit, and wherein the injection device is configured to inject ozone at the feed hopper.

10. The system according to claim 1 wherein the injection device is configured to inject ozone at the intake area of the waste.

11. The system according to claim 1 wherein the injection device is configured to inject ozone at the entry of the tunnel.

12. The system according to claim 1 wherein the conveying device is inclined downward with respect to a horizontal plane in the direction of movement of the waste.

13. The system according to claim 1 wherein the conveying device comprises at least a first conveyor and a second conveyor.

14. The system according to claim 13 wherein the first conveyor and the second conveyor have a height offset H relative to each other such that the waste arriving at the end of travel of the first conveyor drops onto the second conveyor.

15. The system according to claim 1 wherein the heating device comprises at least a plurality of microwave generators and a plurality of associated waveguides configured so that the microwaves generated are distributed along the transport line in the tunnel.

16. A method for pretreating and disinfecting waste, comprising the following steps:
a. moving the waste in a tunnel by a conveying device along a transport line, the tunnel hermetically sealing a part at least of the waste transport line;
b. heating the waste moving in the tunnel by at least one heating device;
c. before or during the two preceding steps, applying ozone on a part at least of the waste moving along the transport line by an ozone injection device,
and comprising at least one step of extracting, from a first area of the tunnel, a portion at least of the atmosphere contained in the tunnel and, after the extraction step, at least one step of reinjecting from a second area of the tunnel said removed portion of the atmosphere.

17. A method according to claim 16 wherein the step of applying ozone is performed before and/or at the same time as the heating step.

18. A method according to claim 16 comprising, before the moving step, a step of stirring by a stirring device.

19. A method according to claim 18 wherein the stirring step is a step of shredding the waste, and wherein the stirring device is a shredding device.
